(19) United States
(12) Patent Application Publication
(10) Pub. No.: US 2011/0008306 A1
(43) Pub. Date: Jan. 13, 2011

(54) CREATINE-LIGAND COMPOUNDS AND METHODS OF USE THEREOF

(76) Inventor: Belinda Tsao NIVAGGIOLI, Atherton, CA (US)

Correspondence Address: MCCARTER & ENGLISH, LLP BOSTON, 265 Franklin Street, Boston, MA, 02110, US

(21) Appl. No.: 12/698,527

(22) Filed: Feb. 2, 2010

Related U.S. Application Data
(62) Division of application No. 11/803,008, filed on May 11, 2007, now abandoned.
(60) Provisional application No. 60/799,744, filed on May 11, 2006.
(60) Provisional application No. 60/922,147, filed on Apr. 6, 2007.

(51) Int. Cl. A61K 31/65; C07C 279/14; C07D 307/33; A61K 31/197; A61P 17/00; A61P 25/00

(52) U.S. Cl. 424/94.1; 562/560; 549/315; 514/565; 514/154

(57) ABSTRACT

The present invention provides methods of treating creatine responsive states, such as a neurological disorder (i.e., Huntington's disease, Parkinson's disease, amyotrophic lateral sclerosis and creatine transporter defect) or a skin disorder, by administering a creatine-ligand compound, alone or in combination with an anti-inflammatory compound, to a subject.

### RELATED APPLICATIONS

This application is a divisional application of U.S. Ser. No. 11/803,008, filed May 11, 2007; which claims priority to U.S. Provisional Patent Application No. 60/922,147, filed on Apr. 6, 2007; and U.S. Provisional Patent Application No. 60/799,744, filed on May 11, 2006. The aforementioned applications are hereby incorporated by reference in their entirety.

### BACKGROUND OF THE INVENTION

Creatine is a naturally occurring compound that is found within the mammalian body, for example, in the brain, heart, retina and skeletal muscle. The lack of creatine in mammalian systems has been implicated in neurological disorders. Neurological disorders are disorders that affect the central nervous system, the peripheral nervous system or the autonomic nervous system. These neurological disorders include, for example, amyotrophic lateral sclerosis (ALS), Huntington's disease, Parkinson's disease and creatine transporter defect.

ALS, often referred to as “Lou Gehrig's disease,” is a progressive neurodegenerative disease that attacks the motor neurons of the brain and spinal cord that are responsible for voluntary muscle movement. As these motor neurons degenerate their ability to send impulses to the muscle fibers is compromised. As the disease progresses, the motor neurons die, which results in the brain's inability to initiate or control muscle movement and, eventually, the patient becomes completely paralyzed and their muscles atrophy. ALS affects roughly 30,000 Americans at one time and every year 5600 new cases of ALS are diagnosed.

Huntington's disease is a progressive neurodegenerative disease caused by a genetic defect. The disease causes the deterioration of neurons in those parts of the brain that are responsible for controlling cognitive, emotional and motor functions. As a result, patients suffer a variety of symptoms including uncontrollable muscle movements, clumsiness, memory loss, and, ultimately, severe mental deterioration. In the United States, approximately 35,000 people suffer from Huntington's disease and another 175,000 people are at risk for developing the disease.

Parkinson's disease is a progressive, neurodegenerative brain disorder that occurs when neurons within the brain that are responsible for producing the chemical dopamine become impaired or die. The cause of this nerve cell damage and death is not completely understood. Eventually, symptoms, which include uncontrolled shaking of the hands and or feet, may progress to a point where routine tasks become severely impaired. It is estimated that approximately 1.5 million Americans are affected by Parkinson's disease, making it the second most common neurodegenerative disease after Alzheimer's disease. Approximately 60,000 new cases are diagnosed each year in the United States.

Creatine transporter defect (CTD) is an inherited error of metabolism that inhibits the body's ability to supply sufficient levels of creatine to the brain via the creatine transporter. Caused by a defect in the X-linked creatine transporter, CTD results in mental retardation with symptoms including speech and language impairment, short attention span and low I.Q.

There are currently no known cures for ALS, Huntington's disease, Parkinson's disease, CTD and many other neurological disorders. Instead, treatment is focused on relieving symptoms, preventing complications and maximizing the quality of life.

In addition, the use of creatine and creatine analogues has been shown to be effective for use in the prevention and treatment of skin disorders, such as free-radicals, aging, sun radiation, stress, fatigue, psoriasis, uneven pigmentation or skin damage.

### SUMMARY OF THE INVENTION

The invention pertains, at least in part, to a composition comprising creatine bound to a ligand to form a creatine-ligand compound in which the creatine-ligand compound has a ratio of between about 1:1 creatine to ligand and about 10:1 creatine to ligand. In one embodiment, the composition further comprises dextrose.

The invention also pertains, at least in part, to a composition comprising about 5 grams of a creatine-ligand compound and a pharmaceutically acceptable carrier having a particle size of between about 1000 and 1500 microns.

In one embodiment, the invention pertains, at least in part, to a method of treating a creatine responsive state in a subject by administering to the subject a composition comprising an effective amount of a creatine-ligand compound such that the creatine responsive state in the subject is treated. Creatine responsive states include, for example, creatine transporter defect, amyotrophic lateral sclerosis, Huntington's disease, Parkinson's disease and skin disorders.

In another embodiment, the invention pertains, at least in part, to a method of treating creatine transporter defect in a subject by administering to the subject an effective amount of a creatine-ligand compound in which the creatine-ligand compound is comprised of between about a 2:1 ratio of creatine to ligand and about an 8:1 ratio of creatine to ligand.

In a further embodiment, the invention pertains, at least in part, to a method of treating amyotrophic lateral sclerosis in a subject by administering to the subject an effective amount of a creatine-ligand compound in which the creatine-ligand compound is comprised of between about a 2:1 ratio of creatine to ligand and about an 8:1 ratio of creatine to ligand. The composition may also include dextrose.

In yet another embodiment, the invention pertains, at least in part, to a method of treating a neurological disorder in a subject by administering to the subject an effective amount of a creatine-ligand compound in combination with an anti-inflammatory compound such that the neurological disorder in said subject is treated.

The invention also pertains, at least in part, to a method of treating amyotrophic lateral sclerosis in a subject by administering to the subject a composition comprising an effective amount of a creatine-ligand compound and dextrose in combination with an anti-inflammatory compound such that the amyotrophic lateral sclerosis in the subject is treated.

In one embodiment, the invention pertains, at least in part, to a method for treatment of a skin disorder by administering an effective amount of a creatine-ligand compound to a subject such that the skin disorder in the subject is treated.

In a further embodiment, the invention pertains, at least in part, to a pharmaceutical composition comprising an effective amount of a creatine-ligand compound and an acceptable carrier, wherein said effective amount is effective for the treatment of a creatine responsive state.

In yet another embodiment, the invention pertains, at least in part, to pharmaceutical composition comprising an effective amount of a creatine-ligand compound in combination with an anti-inflammatory compound, and an acceptable carrier, wherein said effective amount is effective for the treatment of a creatine responsive state.

In another embodiment, the invention pertains, at least in part, to a pharmaceutical composition comprising an effective amount of a creatine-ligand compound and dextrose.

In one embodiment, the present invention pertains, at least in part, to a method for treating amyotrophic lateral sclerosis in a subject by administering to the subject about 5 grams of creatine ascorbate and about 2 grams of dextrose at least once a day.

The present invention pertains, at least in part, to a packaged pharmaceutical composition comprising about 5 grams of creatine ascorbate and about 2 grams of dextrose and instructions for treating amytrophic lateral sclerosis.

### DETAILED DESCRIPTION OF THE INVENTION

### Creatine-Ligand Compounds

The present invention pertains, at least in part, to a composition comprising creatine bound to a ligand to form a creatine-ligand compound, wherein the creatine-ligand compound has a ratio of between about 1:1 creatine to ligand and about 10:1 creatine to ligand.

In another embodiment, the present invention also pertains, at least in part to a composition comprising creatine bound to a ligand to form a creatine-ligand compound and dextrose (also known as α-D-glucose), wherein the creatine-ligand compound has a ratio of between about 1:1 creatine to ligand and about 10:1 creatine to ligand.

Creatine (also known as N-(aminoiminomethyl)-N-methylglycine; methylglycosamine or N-methyl-guanido acetic acid) is a compound of formula (I):

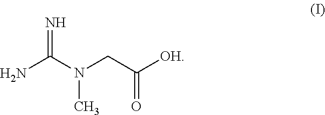

In a further embodiment, the creatine-ligand compound of the invention is pharmaceutical grade. The term “pharmaceutical grade” includes compositions which are substantially free of toxins, such as, but not limited to, creatinine, dihydrotriazine, dicyandiamide, and heavy metals.

Pharmaceutical grade creatine and creatine-ligand compounds are substantially different from the creatine generally available on retail shelves for use as a nutritional supplement. Pharmaceutical grade creatine and creatine-ligand compounds are manufactured under drug GMP guidelines and meets the standards. Unlike nutritional supplement creatine, pharmaceutical grade creatine and creatine ligand compounds are substantially free of neurotoxins or other toxins, which may be dangerous to subjects with neurodegenerative diseases (e.g. Report of the Scientific Committee on Food (SCF), Opinion on safety aspects of creatine supplementation (adopted by the SCF on 7 Sep. 2000).

The term “substantially free of toxins” includes compounds with such low levels of toxins, (e.g., creatinine, dicyandiamide, dihydrotriazine, and heavy metals), that the compounds are appropriate for administration to subjects with neurodegenerative diseases. The levels of toxins can be determined using methods known in the art such as chromatography, e.g., thin layer chromatography. In a further embodiment, the term “substantially free of toxins” includes compositions with less than about 200 ppm, less than about 100 ppm, or less than about 50 ppm of any individual toxin and/or less than about 1000 ppm, less than about 500 ppm, less than about 200 ppm, or less than about 100 ppm of total toxins in the creatine and/or creatine monohydrate.

In contrast to the pharmaceutical grade creatine claimed herein, nutriceutical grade creatine may contain heavy metals such as mercury and lead; about 54,000 ppm (5.4%) dicyandiamide; about 13,000 ppm (1.3%) creatinine; and about 860 ppm dihydroazines.

In one embodiment, the pharmaceutical grade creatine-ligand compounds contain less than about 1% heavy metals, less than about 0.5%, less than about 0.1%, less than about 0.01%, less than about 0.001%, or substantially no heavy metals, such as lead and mercury.

In another further embodiment, the pharmaceutical grade creatine-ligand compounds contain less than about 24,000 ppm, less than about 20,000 ppm, less than about 10,000 ppm, less than about 5,000 ppm, less than about 1,000 ppm, less than about 500 ppm, less than about 100 ppm, less than about 70 ppm, or less than the detectable threshold of dicyandiamide.

In another further embodiment, the pharmaceutical grade creatine-ligand compounds contain less than about 13,000 ppm, less than about 10,000 ppm, less than about 8,000 ppm, less than about 6,000 ppm, less than about 4,000 ppm, less than about 2,000 ppm, less than about 1,000 ppm, less than about 500 ppm, less than about 100 ppm, less than about 50 ppm, less than about 20 ppm, or less than the detectable threshold of creatinine.

In another further embodiment, the pharmaceutical grade creatine-ligand compounds contain less than about 860 ppm, less than about 600 ppm, less than about 400 ppm, less than about 200 ppm, less than about 100 ppm, less than about 50 ppm, or less than the detectable threshold of dihydrotriazines.

The term “ligand,” as used herein, refers to an atom or molecule which binds to creatine through covalent or electrostatic interactions. The term “creatine-ligand compound” refers to a compound in which creatine is bound to another atom or molecule through covalent or electrostatic interactions. The ratio of creatine to the ligand can be, for example, about a 1:1 ratio, about a 2:1 ratio, about a 3:1 ratio, about a 4:1 ratio, about a 5:1 ratio, about a 6:1 ratio, about a 7:1 ratio, about an 8:1 ratio, about a 9:1 ratio or about a 10:1 ratio. The ratio of creatine to the ligand can also be any ratio in which creatine is bound to the ligand through covalent or electrostatic interactions. In one embodiment, the creatine-ligand compound has a ratio of between about 3:1 creatine to ligand and about 6:1 creatine to ligand.

In one embodiment, the ligand is an amino acid. As used herein, the term “amino acid” refers to any molecule that contains both an amino and a carboxylic acid functionality and includes standard and non-standard amino acids. Standard amino acids include, for example, leucine, proline, alanine, valine, glycine, serine, asparagine, glutamine, aspartic acid, glutamic acid, methionine, tryptophan, phenylalanine, isoleucine, threonine, cysteine, tyrosine, histidine, lysine and arginine. Non-standard amino acids include all other amino acids, for example, 5-hydroxylysine, 4-hydroxyproline, thyroxine, 3-methylhistadine, ε-N-methyllysine, ε-N,N,N-trimethyllysine, aminoadipic acid, γ-carboxyglutamic acid, pyroglutamic acid, phosphoserine, phosphotyrosine, N-methylarginine, N-acetyllysine, sarcosine, γ-aminobutyric acid, betaine, β-alanine, azaserine, homoserine, lanthionine, homocysteine, phenylserine, chloramphenicol, cycloserine, epinephrine, histamine, serotonin, penicillamine, ornithine, citrulline and the like.

In one embodiment, the ligand is a water-soluble vitamin. The term “water-soluble vitamin” refers to those vitamins which dissolve easily in water, such as vitamin C (ascorbic acid) and the B-complex vitamins. The B-complex vitamins may include vitamin B₁ (thiamine), B₂ (riboflavin), B₃ (niacin), B₅ (pantothenic acid), B₆ (pyridoxine), B₇ (biotin), B₉ (folic acid), and B₁₂ (cyanocobalamin).

In another embodiment, the ligand is selected from the group consisting of cinnamate, lactate, glycolate, malate, mandelate, ascorbate, phytate, citrate, hydroxycitrate, aleurate, salicylate and hyaluronate. In one particular embodiment, the ligand is ascorbate.

In one embodiment, the creatine-ligand compound with a ratio of between about 2:1 creatine to ligand to about 10:1 creatine to ligand has an increased solubility over a creatine-ligand compound comprised of ratio of about 1:1 creatine to ligand.

In one embodiment, the amount of creatine-ligand compound in the composition is between about 1 gram and about 50 grams. The term “about,” as used with reference to an amount of creatine-ligand compound and/or an anti-inflammatory compound and/or dextrose refers to ±0.5 grams of creatine-ligand compound and/or an anti-inflammatory compound and/or dextrose. In another embodiment, the amount of creatine-ligand compound in the composition is about 1 gram, about 2 grams, about 3 grams, about 4 grams, about 5 grams, about 6 grams, about 7 grams, about 8 grams, about 9, grams, about 10 grams, about 11 grams, about 12 grams, about 13 grams, about 14 grams, about 15 grams, about 16, grams, about 17 grams, about 18 grams, about 19 grams, about 20 grams, about 21 grams, about 22 grams, about 23 grams, about 24 grams, about 25 grams, about 26 grams, about 27 grams, about 28 grams, about 29 grams, about 30 grams, about 31 grams, about 32 grams, about 33 grams, about 34 grams, about 35 grams, about 36 grams, about 37 grams, about 38 grams, about 39 grams, about 40 grams, about 41 grams, about 42 grams, about 43 grams, about 44 grams, about 45 grams, about 46 grams, about 47 grams, about 48 grams, about 49 grams or about 50 grams or greater. In another embodiment, the amount of creatine-ligand compound is a therapeutically effective amount of creatine-ligand compound.

In one embodiment, the amount of dextrose in the composition is between about 1 gram and about 20 grams. In another embodiment, the amount of dextrose in the composition is about 1 gram, about 2 grams, about 3 grams, about 4 grams, about 5 grams, about 6 grams, about 7 grams, about 8 grams, about 9, grams, about 10 grams, about 11 grams, about 12 grams, about 13 grams, about 14 grams, about 15 grams, about 16, grams, about 17 grams, about 18 grams, about 19 grams and about 20 grams. In another embodiment, the amount of dextrose is necessary to enhance the flow characteristics of the composition.

In one embodiment, the particle size of the creatine-ligand compound and/or the dextrose is between about 1000 and about 1500 microns. In another embodiment, the particle size is between about 1100 and 1200 microns. The particle size of the creatine ligand compound and the dextrose is not particularly limited provided that the particle size does not affect the intended function of the composition (e.g., treating a neurological disorder). For example, in one embodiment, the particle size of the creatine-ligand compound and the dextrose is the same. In one particular embodiment, the creatine-ligand compound and/or the dextrose are formulated as a powder.

The creatine-ligand compound may also be mixed with any appropriate carrier. Suitable carriers include any pharmaceutically acceptable carrier (e.g., dextrose). An appropriate carrier can be selected such that it blends well with the creatine-ligand compound (e.g., similar size, consistency or color). The carrier may also be chosen to enhance the flow characteristics of the creatine-ligand compound.

### Creatine Responsive States

In another embodiment, the invention pertains to a method of treating a creatine responsive state in a subject comprising administering to said subject an effective amount of a creatine-ligand compound such that the creatine responsive state in said subject is treated.

As used herein, the term “creatine responsive state” refers to states which can be treated, prevented or otherwise ameliorated by the administration of a creatine-ligand compound of the invention. The language “treating a creatine responsive state” is intended to include prevention of the state, amelioration and/or arrest of a preexisting state, and the elimination of a preexisting state. In one embodiment, the creatine responsive state is a neurological disorder. In another embodiment, the creatine responsive state is a skin disorder.

### 1. Neurological Disorders

In one embodiment, the creatine responsive state is a neurological disorder. In another embodiment, the subject is at risk of suffering from a neurological disorder (e.g., ALS, creatine transporter defect, Huntington's disease, Parkinson's disease). The term “neurological disorder” refers to disorders that may cause a disturbance in the structure or function of the nervous system resulting from developmental abnormalities, disease, genetic defects, injury or toxin. These disorders may affect the central nervous system (e.g., the brain, brainstem and cerebellum), the peripheral nervous system (e.g., the cranial nerves, spinal nerves, and sympathetic and parasympathetic nervous systems) and/or the autonomic nervous system (e.g., the part of the nervous system that regulates involuntary action and that is divided into the sympathetic and parasympathetic nervous systems). Examples of neurological disorders may include, for example, Landau-Bluffer syndrome, acquired epileptiform aphasia, acute disseminated encephalomyelitis, adrenoleukodystrophy, neurological complications of acquired immunodeficiency syndrome (AIDS), Alexander disease, Alper's disease, amyotrophic lateral sclerosis, ataxia, ataxia-telangiectasia, dysautonomia, autonomic dysfunction, familial dysautonomia, Riley-Day syndrome, benign essential blepharospasm, blepharospasm, monomelic amyotrophy, benign focal amyotrophy, Hirayama syndrome, O'Sullivan-McLeod syndrome, subcortical arteriosclerotic encephalopathy, traumatic brain injury, Brown-Sequard syndrome, Kennedy's disease, bulbospinal muscular atrophy, spinal muscular atrophy, Caravan disease, leukodystrophy, central cord syndrome, cerebellar degeneration, cerebral atrophy, Charcot-Marie-Tooth disease, chorea, dyskinesia, Syndenham chorea, neuroacanthcytosis, Levine-Critchley syndrome, choreoacanthocytosis, chronic inflammatory demyelinating polyneuropathy (CIDP), congenital myasthenia, congenital myopathy, central core disease, nemaline rod myopathy, centronuclear (myotubular) myopathy, corticobasal degeneration, Creutzfeldt-Jakob disease, dementia, dyssenergia cerebellaris myoclonica, dyssenergia cerebellaris progressive, dentate cerebellar ataxia, dentatorubral atrophy, primary dentatum atrophy, Ramsey-Hunt syndrome, dermatomytosis, Devic's syndrome, Schilder's disease, myelinoclastic diffuse sclerosis, dystonias, familial periodic paralysis, Friedreich's ataxia, Germann-Straussler-Scheinker disease, Krabbe disease, Guillain-Barre syndrome, hemiplegia alterans, tropical spastic paraparesis, retrovirus-associated myelopathy, HTLV-1 associated myelopathy, Huntington's disease, hypertonia, Isaac's syndrome, neuromyotonia, kuru, opsoclonus myoclonus, Kinsbourne syndrome, spinal muscular atrophy, Werdnig-Hoffman disease, Kugelberg-Welander disease, transmissible spongiform encephalopathies, fatal familial insomnia, Lambert-Eaton myasthenic syndrome, Leigh's disease, locked-in syndrome, Lou Gehrig's disease, lupus, systemic lupus erythematosus, Machado-Joseph disease, Melkersson-Rosenthal syndrome, Miller Fisher syndrome, mitochondrial myopathies, motor neuron disease, multifocal motor neuropathy, multiple sclerosis, multiple system atrophy, muscular dystrophy, myasthenia gravis, neurofibromatotis, von Recklinghausen's disease, neurological complications of Lyme's disease, thyrotoxic myopathy, tabes dorsalis, progressive locomotor ataxia, prion diseases, primary lateral sclerosis, acute demyelinating neuropathy, acute disseminated encephalomyelitis, acute necrotizing hemorrhagic leukoencephalitis, metachromic leukodystrophy, adrenoleukodystrophy, adrenomyeloneuropathy, spinocerebellar degenerations, mitochondrial encephalomyopathies, Pelizaeus-Merzbacher disease, creatine transporter defect, and Duchenne muscular dystrophy. In one embodiment, the neurological disorder is Huntington's disease, Parkinson's disease, amyotrophic lateral sclerosis or creatine transporter defect.

The term “treating a neurological disorder” is intended to include prevention of the disorder, amelioration and/or arrest of a preexisting disorder, and the elimination of a preexisting disorder, or the prevention, amelioration and/or arrest of one or more symptoms of the neurological disorder.

In one embodiment, the invention pertains, at least in part, to methods of treating creatine transporter defect in a subject in which an effective amount of a creatine-ligand compound is administered to the subject, wherein the creatine-ligand compound is comprised of between about a 4:1 ratio of creatine to ligand to about an 8:1 ratio of creatine to ligand. In another embodiment, the invention pertains, at least in part to methods of treating creatine transporter defect in a subject in which an effective amount of a creatine-ligand compound and dextrose is administered to the subject, wherein the creatine-ligand compound is comprised of between about a 2:1 ratio of creatine to ligand to about an 8:1 ratio of creatine to ligand. In one particular embodiment, the method ligand is ascorbate.

The term “creatine transporter defect” includes a disorder characterized by an inborn error creatine synthesis or of the creatine transporter or other aberrant creatine transport function in the brain. The aberrant creatine transport function in the brain may cause the subject to suffer from a low concentration of creatine in the brain of a subject suffering from creatine transporter dysfunction. In this disorder, impaired energy metabolism is believed to be associated with impaired learning dysfunction and cognitive function. It was found that treatments of similar neurological or cognitive dysfunctions do not tend to target improving metabolism and/or energy metabolism of the brain, neural cells, or glial cells.

In yet another embodiment, the invention pertains, at least in part, to a method of treating amyotrophic lateral sclerosis in a subject comprising administering to the subject an effective amount of a creatine-ligand compound, wherein the creatine-ligand compound is comprised of between about a 2:1 ratio of creatine to ligand and about an 8:1 ratio of creatine to ligand. In one embodiment, the creatine-ligand compound is comprised of about a 2:1 ratio of creatine to ligand. In one particular embodiment, the ligand is ascorbate.

In one embodiment, the invention also pertains to a method of treating amyotrophic lateral sclerosis in a subject by administering to the subject a composition comprising an effective amount of a creatine-ligand compound and dextrose in combination with an anti-inflammatory compound such that the amyotrophic lateral sclerosis in said subject is treated.

In yet another embodiment, the invention pertains, at least in part, to a method of treating amyotrophic lateral sclerosis in a human by administering to the human a composition comprising a creatine-ligand compound (e.g., creatine ascorbate) and dextrose. In one embodiment, the method pertains to treating amyotrophic lateral sclerosis in a human by administering to the human a comprising between about 1 gram and about 50 grams of a creatine-ligand compound (e.g., about 5 grams of creatine ascorbate) and between about 1 gram and 20 grams of dextrose (e.g., about 2 grams). The composition may be administered to the subject for treatment of amyotrophic lateral sclerosis, for example, in the amount of 5 grams of creatine ascorbate and 2 grams of dextrose at least once a day.

In yet a further embodiment, the invention pertains, at least in part, to a method for treating amyotrophic lateral sclerosis in a subject comprising administering to the subject about 5 grams of creatine ascorbate and about 2 grams of dextrose at least once a day.

In one embodiment, the compositions of the invention slow or prevent cortical thinning of the brain. The term “cortical thinning” refers to the decrease in the mass, thickness and/or surface area of the brain.

In another embodiment, the compositions of the invention modulate the levels of one or more biomarkers in the subject. The term “biomarker” includes any molecular species found to provide correlation to a particular phenotype or perturbation of a biological system and is used to indicate or measure a biological process (e.g., a neurological disorder). In one embodiment, the biomarker is serum 8-hydroxy-2′-deoxyguanosine (80H2′dG). In a further embodiment, the level of 80H2′dG in a subject suffering from or at risk of suffering from a neurological disorder is reduced to a level comparable to the level of 80H2′dG in a healthy subject.

In yet another embodiment, the compositions of the invention modulate the levels of one or more genetic markers in said subject. The term “genetic marker” includes known DNA sequences that can be identified by a simple assay, for example, a short DNA sequence, such as a sequence surrounding a single base-pair change (single nucleotide polymorphism), or a long one DNA sequence, such as a microsatellite. Genetic markers can be used to study the relationship between a disease and its genetic cause (for example, a particular mutation of a gene that results in a defective protein). In an embodiment, the genetic marker is pleopmorphic adenoma gene-like 1 (PLAG1) or H2A histone family, member Y (H2AFY).

The term “modulate” includes, for example, the increase or decrease of the concentration of biomarker or the genetic marker found in a subject suffering from or at risk of suffering from a neurological disorder.

In another embodiment, the present invention pertains, at least in part, to methods of treating a neurological disorder in a subject in which an effective amount of a creatine-ligand compound in combination with an anti-inflammatory compound is administered to the subject. In one particular embodiment, the creatine-ligand compound is administered with dextrose.

The term “anti-inflammatory compound” refers compounds that treat, prevent or ameliorate inflammation in a subject. The anti-inflammatory compounds of the present invention include, for example, members of the tetracycline family, opiate agonists, lipoxygenase inhibitors, cyclooxygenase inhibitors (e.g., cyclooxygenase-1 (COX-1) selective inhibitors, cyclooxygenase-2 (COX-2) selective inhibitors and non-selective cyclooxygenase inhibitors), interleukin receptor antagonists, NMDA receptor antagonists, inhibitors of nitric oxide or inhibitors of the synthesis of nitric oxide, non-steroidal anti-inflammatory agents, steroidal anti-inflammatory compounds or cytokine-suppressing anti-inflammatory agents

In one embodiment, the anti-inflammatory compound may be a member of the tetracycline family. The language “member of the tetracycline family” includes many compounds with a similar ring structure to tetracycline. Examples of tetracycline compounds include: oxytetracycline, demeclocycline, methacycline, minocycline, sancycline, chelocardin, rolitetracycline, lymecycline, apicycline; clomocycline, guamecycline, meglucycline, mepylcycline, penimepicycline, pipacycline, etamocycline, penimocycline, etc. Other derivatives and analogues comprising a similar four ring structure are also included (See Rogalski, “Chemical Modifications of Tetracyclines,” the entire contents of which are hereby incorporated herein by reference). Table 1 depicts tetracycline and several known members of the tetracycline family.

TABLE 1 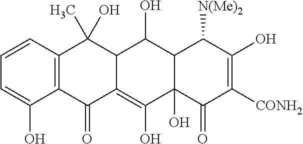
Oxytetracycline 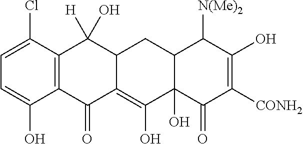
Demeclocycline 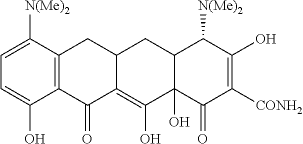
Minocycline 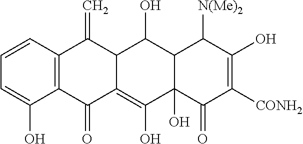
Methacycline 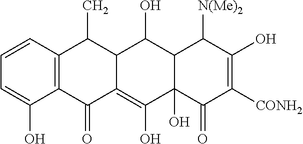
Doxycycline 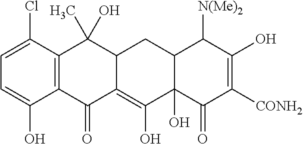
Chlortetracycline 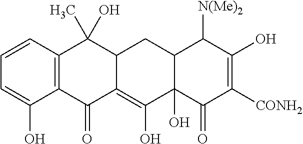
Tetracycline 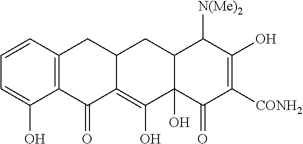
Sancycline 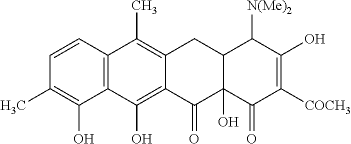
Chelocardin

In one embodiment, the member of the tetracycline compound is selected from the group consisting of oxytetracycline, demeclocycline, minocycline, methacycline, doxycycline, chlortetracycline, tetracycline, sancycline, chelocardin and pharmaceutically acceptable derivatives thereof. In one embodiment, the member of the tetracycline family is minocycline. The dosage of the member of the tetracycline family may be between about 50 and 500 mg per day. In one embodiment, the dosage is 400 mg. In another embodiment, the dosage is about 100 mg per day.

In another embodiment, the invention pertains, at least in part, to a method for treating amyotrophic lateral sclerosis in a subject comprising administering to the subject about 5 grams of creatine ascorbate and about 2 grams of dextrose in combination with minocycline at least once a day.

In another embodiment, the anti-inflammatory may be a cyclooxygenase-2 (COX-2) selective inhibitor. The language “cyclooxygenase-2 (COX-2) selective inhibitor” refers to compounds that selectively inhibit the cyclooxygenase-2 enzyme over the cyclooxygenase-1 enzyme. Suitable COX-2 inhibitors include, for example, 2-(4-ethoxy-phenyl)-3-(4-methanesulfonyl-phenyl)-pyrazolo[1,5-b]pyridazine, CDC-501, celecoxib, COX-189, CS-179, CS-502, D-1367, 4-(2-oxo-3-phenyl-2,3-dihydrooxazol-4-yl) benzenesulfonamide, darbufelone, DFP, DRF-4367, etodolac, flosulide, JTE-522 (4-(4-cyclohexyl-2-methyl-5-oxazolyl)-2-fluorobenzenesulfonamide), L-745337, L-748731, L-748780, L-768277, L-776967, L-783003, L-791456, L-804600, L-748706, meloxicam, MK663 (etoricoxib), nimesulide, NS-398, parecoxib, 1-methylsulfonyl-4-(1,1-dimethyl-4-(4-fluorophenyl)cyclopenta-2,4-dien-3-yl)benzene, DuP-697, L-761066, 4-(1,5-dihydro-6-fluoro-7-methoxy-3-(trifluoromethyl)-(2)-benzothiopyrano-(4,3-c)pyrazol-1-yl)benzenesulfonamide, 4,4-dimethyl-2-phenyl-3-(4-methylsulfonyl)phenyl)cyclobutenone, 4-amino-N-(4-(2-fluoro-5-trifluoromethyl)-thiazol-2-yl)benzene sulfonamide, meloxicam, 1-(7-tert-butyl-2,3-dihydro-3,3-dimethyl-5-benzo-furanyl)-4-cyclopropyl-butan-1-one, rofecoxib, RS-113472, RWJ-63556, RS-57067, S-2474, S-33516, SC-299, SC-5755, SC-57666, SC-58125, lumiracoxib valdecoxib, parecoxib sodium, APHS, UR-8877, UR-8813, UR-8880 and pharmaceutically acceptable derivatives thereof. In one embodiment, the COX-2 selective inhibitor is celecoxib. In another embodiment, the dosage of the COX-2 selective inhibitor is between about 100 and 500 mg per day. In a further embodiment, the dosage of the COX-2 selective inhibitor is about 400 mg per day.

In another embodiment, the invention pertains, at least in part, to a method for treating amyotrophic lateral sclerosis in a subject comprising administering to the subject about 5 grams of creatine ascorbate and about 2 grams of dextrose in combination with celecoxib at least once a day.

In one embodiment, the method of treating a neurological disorder in a subject further comprises administering to the subject an effective amount of one or more therapeutic agents including, but not limited to, co-enzyme Q₁₀, ethyl-eicosapentanopic acid, a glutamate antagonist (e.g., riluzole, lamotrigine or remacemide) or phenylbutyrate.

The language “in combination with” an anti-inflammatory compound includes co-administration of the creatine-ligand compound and with an anti-inflammatory compound, administration of the creatine-ligand compound first, followed by administration of an anti-inflammatory compound, and administration the anti-inflammatory compound first, followed by administration of the creatine-ligand compound. The creatine-ligand compound can be administered substantially at the same time as the anti-inflammatory compound or at substantially different times as the anti-inflammatory compounds. Optimal administration rates for a given protocol of administration of the creatine-ligand compound and/or the anti-inflammatory compound can be readily ascertained by those skilled in the art using conventional dosage determination tests conducted with regard to the specific compounds being utilized, the particular compositions formulated, the mode of application, the particular site of administration and the like.

### 2. Skin Disorders

In another embodiment, the creatine responsive state is a skin disorder. Examples of skin disorder include, but are not limited to, aging, damage resulting from sun radiation, stress, uneven pigmentation, psoriasis, fatigue and/or damage associated with free radicals. In another embodiment, the subject is at risk of suffering from a skin disorder. In a further embodiment, the subject is afflicted with skin wrinkles. The language “treating for skin disorders” includes both prevention of disorders, amelioration and/or arrest of the disorder process. The language also includes any amelioration or arrest of any symptoms associated with the disorder process (e.g., wrinkles). For example, treating wrinkles may include preventing, retarding, arresting, or reversing the process of wrinkle formation in skin, e.g., mammalian skin, preferably, human skin.

The term “aging” includes processes where there is oxidative damage, energy depletion or mitochondrial dysfunction where onset, amelioration, arrest, or elimination is effectuated by the creatine compounds described herein. Symptoms of aging include, but are not limited to, wrinkles, loss of elasticity of the skin and uneven pigmentation of the skin.

The term “associated with free radicals” includes any disorders or damaged to the skin resulting from, directly or indirectly from free radicals. The free radicals may be initiated by, for example, sun radiation (e.g., UV radiation) or pollution.

In one embodiment, the invention pertains, at least at part, a method of treating a skin disorder in which an effective amount of a creatine-ligand compound is administered to a subject such that the skin disorder in said subject is treated. In one embodiment, the treatment of the skin disorder reduces or eliminates at least one preexisting symptom of the skin disorder. The preexisting symptom may include, for example, skin wrinkles or a loss of skin elasticity. In another embodiment, the treatment of the skin disorder comprises the prevention of the skin disorder.

In yet another embodiment the method of treating a skin disorder further comprises co-administering to a subject an effective amount of a creatine-ligand compound and an effective amount of a skin preserving agent. Examples of skin preserving agents include antioxidants, such as ascorbic acid, vitamins, coenzyme Q10 (CoQ10) and its derivatives, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like. Preferred anti-oxidants include, CoQ10 and vitamin E. Other examples of skin preserving agents include energy-enhancing agents (e.g., ATP, nicotinamide or pyruvate), vitamins (e.g., E, C, B5, B6, and B9) and vitamin precursors.

The term “energy enhancing agents” also includes stimulants of mitochondrial function or ATP production elsewhere in the cell. Examples include intermediates such as, for example, pyruvate, nicotinamide and CoQ10.

The term “subject” is intended to include living organisms susceptible to having creatine responsive states (e.g., mammals). Examples of subjects include humans, dogs, cats, horses, cows, goats, rats and mice. The term “subject” also includes include transgenic species. In one embodiment, the subject is a human.

### Pharmaceutical Compositions

In one embodiment, the invention pertains, at least in part, to a pharmaceutical composition comprising an effective amount of a creatine-ligand compound and an acceptable carrier, wherein said effective amount is effective for the treatment of a creatine responsive state. In another embodiment, the invention pertains, at least in part, to a pharmaceutical composition comprising an effective amount of a creatine-ligand compound in combination with an anti-inflammatory compound, and an acceptable carrier, wherein said effective amount is effective for the treatment of a creatine responsive state. In one embodiment, the acceptable carrier is suitable for oral or topical administration.

The phrase “acceptable carrier” includes a pharmaceutically or cosmetically acceptable material, composition or vehicle, such as a liquid or solid filler, diluent, excipient, solvent or encapsulating material, involved in carrying or transporting the creatine-ligand compound and/or an anti-inflammatory compound within or to the subject such that it can performs its intended function. Typically, such compounds are carried or transported from one organ, or portion of the body, to another organ, or portion of the body. Each carrier must be “acceptable” in the sense of being compatible with the other ingredients of the formulation and not injurious to the subject. Some examples of materials which can serve as pharmaceutically or cosmetically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical or cosmetic formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically and cosmetically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

The pharmaceutical compositions of the present invention may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Those skilled in the art related to the present invention will be better able to determine an appropriate dosage and overall dosage regime when taking a number of factors into consideration. For example, the size, weight and condition of the patient must be considered as must be the responsiveness of the patient and their disorder to the particular therapy. In one embodiment, the dosage is from 0.001 μg to 100 g and may be administered once or several times daily, weekly, monthly or yearly, or even every 2 to 20 years. In one embodiment, a suitable dose of a compound of the invention will be that amount of the compound which is the lowest dose effective to produce a therapeutic effect. In another embodiment, a suitable dose of a compound of the invention will be an effective daily dose, which includes the lowest daily dose effective to produce a therapeutic effect. The effective daily dose of the active compound may be administered as two, three, four, five, six or more sub-doses administered separately at appropriate intervals throughout the day, optionally, in unit dosage forms.

The language “effective amount” of the compound is that amount necessary or sufficient to treat, prevent or ameliorate a creatine responsive state in a subject. The effective amount can vary depending on such factors as the size and weight of the subject, the type of illness, etc. One of ordinary skill in the art would be able to study the aforementioned factors and make the determination regarding the effective amount of the creatine-ligand compound and/or the anti-inflammatory compound without undue experimentation.

In one embodiment, the effective amount of the creatine-ligand compound is a dosage of the creatine-ligand compound. The term “dosage of creatine-ligand compound” refers to a specified quantity of the creatine-ligand compound. In one embodiment, the dosage of the creatine-ligand compound is between about 5 grams and about 50 grams. The term “about,” as used with reference to a dosage of the creatine-ligand compound and/or an anti-inflammatory compound refers to ±0.5 grams of the creatine-ligand compound and/or an anti-inflammatory compound. In another embodiment, the dosage of the creatine-ligand compound is about 5 grams, about 6 grams, about 7 grams, about 8 grams, about 9, grams, about 10 grams, about 11 grams, about 12 grams, about 13 grams, about 14 grams, about 15 grams, about 16, grams, about 17 grams, about 18 grams, about 19 grams, about 20 grams, about 21 grams, about 22 grams, about 23 grams, about 24 grams, about 25 grams, about 26 grams, about 27 grams, about 28 grams, about 29 grams, about 30 grams, about 31 grams, about 32 grams, about 33 grams, about 34 grams, about 35 grams, about 36 grams, about 37 grams, about 38 grams, about 39 grams, about 40 grams, about 41 grams, about 42 grams, about 43 grams, about 44 grams, about 45 grams, about 46 grams, about 47 grams, about 48 grams, about 49 grams or about 50 grams or greater. In another embodiment, the dosage of the creatine-ligand compound is a therapeutically effective amount of the creatine-ligand compound.

In one embodiment, the dosage of the creatine-ligand compound for the treatment of a creatine-responsive state is between about 5 grams and 50 grams. In another embodiment, the dosage of the creatine-ligand compound for the treatment of a creatine-responsive state is about 40 grams. In another embodiment, the creatine-ligand compound in said dosage is comprised of between about a 1:1 ratio of creatine to ligand and a 10:1 ratio of creatine to ligand. In one embodiment, the creatine-ligand compound in said dosage is comprised of about a 3:1 ratio of creatine to ligand. In a further embodiment, the ligand is ascorbate.

In yet another embodiment, the effective amount of the creatine-ligand compound for the treatment of amyotrophic lateral sclerosis is a dosage of between about 5 grams and 50 grams of the creatine-ligand compound. In another embodiment, the dosage of the creatine-ligand compound for the treatment of amyotrophic lateral sclerosis is about 40 grams. In another embodiment, the creatine-ligand compound in the dosage is comprised of between about a 1:1 ratio of creatine to ligand and a 10:1 ratio of creatine to ligand. In one embodiment, the creatine-ligand compound in said dosage is comprised of about a 3:1 ratio of creatine to ligand. In a further embodiment, the ligand is ascorbate.

1. Oral, Nasal, Transdermal, Buccal, Sublingual and/or Parenteral Administration

In one embodiment, formulations of the invention include those suitable for oral, nasal, transdermal, buccal, sublingual and/or parenteral administration. The formulations may conveniently be presented in unit dosage form and may be prepared by any methods well known in the art of pharmacy. Methods of preparing these formulations or compositions include the step of bringing into association the creatine-ligand compound and/or an anti-inflammatory compound, with the carrier and, optionally, one or more accessory ingredients. In general, the formulations are prepared by uniformly and intimately bringing into association a compound of the invention with liquid carriers, or finely divided solid carriers, or both, and then, if necessary, shaping the product.

Formulations of the invention suitable for oral administration may be in the form of capsules, cachets, pills, wafers, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention as an active ingredient. A compound of the invention may also be administered as a bolus, electuary or paste.

In solid dosage forms of the invention for oral administration (capsules, tablets, wafers, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the creatine-ligand compound and/or an anti-inflammatory compound include pharmaceutically acceptable emulsions, microemulsions, solutions, suspensions, syrups and elixirs. In addition to the active ingredient, the liquid dosage forms may contain inert dilutents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert dilutents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for transdermal administration of compounds of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required. The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the creatine-ligand compound and/or an anti-inflammatory compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of the creatine-ligand compound and/or an anti-inflammatory compound to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Pharmaceutical compositions of this invention suitable for parenteral administration comprise the creatine-ligand compound and/or an anti-inflammatory compound in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a compound, it is desirable to slow the absorption of the compound from subcutaneous or intramuscular injection. This may be accomplished by the use of a liquid suspension of crystalline or amorphous material having poor water solubility. The rate of absorption of the compound then depends upon its rate of dissolution which, in turn, may depend upon crystal size and crystalline form. Alternatively, delayed absorption of a parenterally-administered compound form is accomplished by dissolving or suspending the compound in an oil vehicle.

Injectable depot forms are made by forming microencapsule matrices of the compounds of the invention in biodegradable polymers such as polylactide-polyglycolide. Depending on the ratio of compound to polymer, and the nature of the particular polymer employed, the rate of compound release can be controlled. Examples of other biodegradable polymers include poly(orthoesters) and poly(anhydrides). Depot injectable formulations are also prepared by entrapping the drug in liposomes or microemulsions which are compatible with body tissue.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systematically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

The creatine-ligand compound and/or an anti-inflammatory compound may be administered to humans and other animals for therapy by any suitable route of administration, including orally, nasally, as by, for example, a spray, parenterally, intracisternally and topically, as by powders, ointments or drops, including buccally and sublingually.

### 2. Topical Administration

In one embodiment, the acceptable carrier is suitable for topical administration. The creatine-ligand compound and/or the anti-inflammatory may be suitable for administration as a lotion, cream, mousse, aerosol, gel, emulsion, solution, ointment, or medicated pad.

The topical pharmaceutical compositions of the present invention formulated as solutions typically include a pharmaceutically-acceptable aqueous or organic solvent. The terms “pharmaceutically-acceptable aqueous solvent” and “pharmaceutically-acceptable organic solvent” refer to a solvent which is capable of having dispersed or dissolved therein the active compound, and possesses acceptable safety properties (e.g., irritation and sensitization characteristics). Water is a typical aqueous solvent. Examples of suitable organic solvents include: propylene glycol, butylene glycol, polyethylene glycol (200-600), polypropylene glycol (425-2025), glycerol, 1,2,4-butanetriol, sorbitol esters, 1,2,-6-hexanetriol, ethanol, isopropanol, butanediol, and mixtures thereof. Preferably, these solutions contain from about 0.01% to about 50% of the active compound, more preferably from about 0.1% to about 20%; and from about 1% to about 80% of an acceptable aqueous or organic solvent, more preferably from about 1% to about 40%.

If the topical pharmaceutical compositions of the present invention are formulated as an aerosol and applied to the skin as a spray-on, a propellant is added to a solution composition. A more complete disclosure of propellants useful herein can be found in Sagarin, Cosmetics Science and Technology, 2nd Edition, Vol. 2, pp. 443-465 (1972).

Topical pharmaceutical compositions of the present invention may be formulated as a solution comprising an emollient. An example of a composition formulated in this way would be a sunscreen-containing product. Preferably, such compositions contain from about 0.1% to about 50% of the active compound and from about 2% to about 50% of a topical pharmaceutically-acceptable emollient.

As used herein, “emollients” refer to materials used for the prevention or relief of dryness, as well as for the protection of the skin. A wide variety of suitable emollients is known and may be used herein. Sagarin, Cosmetics, Science and Technology, 2nd Edition, Vol. 1, pp. 32-43 (1972), incorporated herein by reference, contains numerous examples of suitable materials.

A lotion can be made from a solution carrier system. Lotions preferably comprise from about 0.1% to about 20%, more preferably from about 1% to about 5%, of the active compound; from about 1% to about 20%, preferably from about 5% to about 10%, of an emollient; and from about 50% to about 90%, preferably from about 60% to about 80%, water.

Another type of product that may be formulated from a solution carrier system is a cream. A cream of the present invention would preferably comprise from about 0.1% to about 20%, more preferably from about 1% to about 5%, of the active compound; from about 5% to about 50%, preferably from about 10% to about 20%, of an emollient, and from about 45% to about 85%, preferably from about 50% to about 75%, water.

Yet another type of product that may be formulated from a solution carrier system is an ointment. An ointment may comprise a simple base of animal or vegetable oils or semi-solid hydrocarbons (oleaginous). Ointments may also comprise absorption ointment bases which absorb water to form emulsions. Ointment carriers may also be water soluble. An ointment may also comprise from about 2% to about 10% of an emollient plus from about 0.1% to about 2% of a thickening agent. A more complete disclosure of thickening agents useful herein can be found in Segarin, Cosmetics, Science and Technology, 2nd Edition, Vol. 1, pp. 72-73 (1972).

If the carrier is formulated as an emulsion, from about 1% to about 10%, preferably from about 2% to about 5%, of the carrier system comprises an emulsifier. Emulsifiers may be nonionic, anionic or cationic. Suitable emulsifiers are disclosed in, for example, U.S. Pat. No. 3,755,560, issued Aug. 28, 1973, Dickert et al; U.S. Pat. No. 4,421,769, issued Dec. 20, 1983, Dixon et al.; and McCutcheon's Detergents and Emulsifiers, North American Edition, pages 317-324 (1986); the disclosures of which are incorporated herein by reference. Preferred emulsifiers are anionic or nonionic, although the other types may also be used.

Lotions and creams can be formulated as emulsions as well as solutions. Preferably such lotions comprise from about 0.1% to about 20%, more preferably from about 1% to about 5%, of the active compound; from about 1% to about 20%, preferably from about 5% to about 10%, of an emollient; from about 25% to about 75%, preferably from about 45% to about 95%, water; and from about 0.1% to about 10%, preferably from about 0.5% to about 5%, of an emulsifier. Such creams would preferably comprise from about 0.1% to about 20%, more preferably from about 1% to about 5%, of the active compound; from about 1% to about 20%, preferably from about 5% to about 10%, of an emollient; from about 20% to about 80%, preferably from about 30% to about 70%, water; and from about 1% to about 10%, preferably from about 2% to about 5%, of an emulsifier.

Single emulsion skin care preparations, such as lotions and creams, of the oil-in-water type and water-in-oil type are well-known in the cosmetic art and are useful in the present invention. Multiphase emulsion compositions, such as the water-in-oil-in-water type, as disclosed in U.S. Pat. No. 4,254,105, Fakuda et al., issued Mar. 3, 1981, incorporated herein by reference, are also useful in the present invention. In general, such single or multiphase emulsions contain water, emollients and emulsifiers as essential ingredients.

Triple emulsion carrier systems comprising an oil-in-water-in-silicone fluid emulsion composition as disclosed in U.S. Pat. No. 4,960,764, Figueroa, issued Oct. 2, 1990, are also useful in the present invention. Preferably, this triple emulsion carrier system can be combined with from about 0.1% to about 20%, more preferably from about 1% to about 5%, of the active compound to yield the topical pharmaceutical composition of the present invention.

Another emulsion carrier system useful in the topical pharmaceutical compositions of the present invention is a micro-emulsion carrier system. Such a system comprises from about 9% to about 15% squalane; from about 25% to about 40% silicone oil; from about 8% to about 20% of a fatty alcohol; from about 15% to about 30% of polyoxyethylene sorbitan mono-fatty acid (commercially available under the trade name Tweens) or other nonionics; and from about 7% to about 20% water. This carrier system is preferably combined with from about 1% to about 5% of the active compound.

If the topical pharmaceutical compositions of the present invention are formulated as a gel or a cosmetic stick, a suitable amount of a thickening agent, as disclosed supra, is added to a cream or lotion formulation.

The topical pharmaceutical compositions of the present invention may also be formulated as makeup products such as foundations.

The topical pharmaceutical compositions of the present invention may also be formulated as medicated pads. Suitable examples of these pads are fully disclosed in U.S. Pat. Nos. 4,891,227 and 4,891,228, to Thaman et al., both issued Jan. 2, 1990 the disclosures of which are incorporated herein.

The topical pharmaceutical compositions of the present invention may contain, in addition to the aforementioned components, a wide variety of additional oil-soluble materials and/or water-soluble materials conventionally used in topical compositions, at their art-established levels.

Various water-soluble materials may also be present in the compositions of this invention. These include humectants, proteins and polypeptides, preservatives and an alkaline agent. In addition, the topical compositions herein can contain conventional cosmetic adjuvants, such as dyes, opacifiers (e.g., titanium dioxide), pigments and perfumes.

The topical pharmaceutical compositions of the present invention may also include a safe and effective amount of a penetration enhancing agent. A preferred amount of penetration enhancing agent is from about 1% to about 5% of the composition. Another useful penetration enhancer for the present invention is the nonionic polymer under the CTFA designation: polyacrylamide and isoparrafin and laureth-7, available as Sepigel from Seppic Corporation. Also useful is polyquaternium-32 and mineral oil known as SalCare SC92 available from Allied Colloids, Suffolk, Va. This is a class of cationic polymers which are generally described in U.S. Pat. No. 4,628,078 to Glover et al. issued Dec. 9, 1986 and U.S. Pat. No. 4,599,379 to Flesher et al. issued Jul. 8, 1986 both of which are incorporated by reference herein.

Examples of useful penetration enhancers, among others, are disclosed in U.S. Pat. Nos. 4,537,776, Cooper, issued Aug. 27, 1985; 4,552,872, Cooper et al., issued Nov. 12, 1985; 4,557,934, Cooper, issued Dec. 10, 1985; 4,130,667, Smith, issued Dec. 19, 1978; 3,989,816, Rhaadhyaksha, issued Nov. 2, 1976; 4,017,641, DiGiulio, issued Apr. 12, 1977; and European Patent Application 0043738, Cooper et al., published Jan. 13, 1982.

Other conventional skin care product additives may also be included in the compositions of the present invention. For example, collagen, hyaluronic acid, elastin, hydrolysates, primrose oil, jojoba oil, epidermal growth factor, soybean saponins, mucopolysaccharides, and mixtures thereof may be used.

Various vitamins may also be included in the compositions of the present invention. For example, Vitamin A, ascorbic acid, Vitamin B, biotin, panthothenic acid, Vitamin D, Vitamin E and mixtures thereof and derivatives thereof are contemplated.

Also contemplated are skin cleaning compositions comprising both active compounds of the present invention and a cosmetically-acceptable surfactant. The term “cosmetically-acceptable surfactant” refers to a surfactant which is not only an effective skin cleanser, but also can be used without undue toxicity, irritation, allergic response, and the like. Furthermore, the surfactant must be capable of being commingled with the active compound in a manner such that there is no interaction which would substantially reduce the efficacy of the composition for regulating skin damage, e.g., wrinkles.

The skin cleaning compositions of the present invention preferably contain from about 0.1% to about 20%, preferably from about 1% to about 5%, of the creatine compound (e.g., creatine, cyclocreatine or another creatine compound) and from about 1% to about 90%, more preferably from about 1% to about 10%, of a cosmetically-acceptable surfactant.

The physical form of the skin cleansing compositions is not critical. The compositions can be, for example, formulated as toilet bars, liquids, pastes, mousses, or pads.

The surfactant component of the compositions of the present invention are selected from anionic, nonionic, zwitterionic, amphoteric and ampholytic surfactants, as well as mixtures of these surfactants. Such surfactants are well-known to those skilled in the detergency art.

The cleaning compositions of the present invention can optionally contain, at their art-established levels, materials which are conventionally used in skin cleansing compositions.

Sunblocks and sunscreens incorporating creatine compounds are also contemplated. The term “sun block” or “sun screen” includes compositions which block UV light. Examples of sunblocks include, for example, zinc oxide and titanium dioxide.

Sun radiation is one cause major cause of skin damage, e.g., wrinkles. Thus, for purposes of wrinkle treatment or prevention, the combination of creatine compounds with a UVA and/or UVB sunscreen would be advantageous. The inclusion of sunscreens in compositions of the present invention will provide immediate protection against acute UV damage. Thus, the sunscreen will prevent further skin damage caused by UV radiation, while the compounds of the invention regulates existing skin damage.

A wide variety of conventional sunscreening agents are suitable for use in combination with the active compound. Segarin, et al., at Chapter VIII, pages 189 et seq., of Cosmetics Science and Technology, disclose numerous suitable agents. Specific suitable sunscreening agents include, for example: p-aminobenzoic acid, its salts and its derivatives (ethyl, isobutyl, glyceryl esters; p-dimethylaminobenzoic acid); anthranilates (i.e., o-aminobenzoates; methyl, menthyl, phenyl, benzyl, phenylethyl, linalyl, terpinyl, and cyclohexenyl esters); salicylates (amyl, phenyl, benzyl, menthyl, glyceryl, and dipropyleneglycol esters); cinnamic acid derivatives (methyl and benzyl esters, .alpha.-phenyl cinnamonitrile; butyl cinnamoyl pyruvate); dihydroxycinnamic acid derivatives (umbelliferone, methylumbelliferone, methylaceto-umbelliferone); trihydroxycinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); dihydroxy-naphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and vilouric acids; tannic acid and its derivatives (e.g., hexaethylether); (butyl carbotol) (6-propyl piperonyl)ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-iso-propyldibenzoylmethane; butylmethoxydibenzoylmethane; etocrylene; and 4-isopropyl-di-benzoylmethane.

Preferred sunscreens useful in the compositions of the present invention are 2-ethylhexyl-p-methoxycinnamate, butylmethoxydibenzoylmethane, 2-hydroxy-4-methoxybenzophenone, octyldimethyl-p-aminobenzoic acid and mixtures thereof.

A safe and effective amount of sunscreen may be used in the compositions of the present invention. The sunscreening agent must be compatible with the active compound. Generally the composition may comprise from about 1% to about 20%, preferably from about 2% to about 10%, of a sunscreening agent. Exact amounts will vary depending upon the sunscreen chosen and the desired Sun Protection Factor (SPF).

Also particularly useful in the present invention are sunscreens such as those disclosed in Sabatelli, U.S. patent application Ser. No. 054,085 (filed Jun. 2, 1987) and Sabatelli et al., U.S. patent application Ser. No. 054,046 (filed Jun. 2, 1987). The sunscreening agents disclosed therein have, in a single molecule, two distinct chromophore moieties which exhibit different ultra-violet radiation absorption spectra. One of the chromophore moieties absorbs predominantly in the UVB radiation range and the other absorbs strongly in the UVA radiation range.

An agent may also be added to any of the compositions of the present invention to improve the skin substantivity of those compositions, particularly to enhance their resistance to being washed off by water, or rubbed off. A preferred agent which will provide this benefit is a copolymer of ethylene and acrylic acid. Compositions comprising this copolymer are disclosed in U.S. Pat. No. 4,663,157, Brock, issued May 5, 1987, which is incorporated herein by reference.

### EXEMPLIFICATION OF THE INVENTION

### Example 1

### Effect of Creatine Ascorbate on Huntington's Disease

Methods

Study design. Sixty-four subjects are enrolled at four sites. Eligible subjects are randomized to 8 g/day of creatine ascorbate or placebo by computer-generated blocked randomization with site stratification. Treatment is administered as chewable wafers twice daily for 16 weeks. A medical monitor and independent safety committee are reviewing clinical data monthly. Consent, study procedures, and case report forms are approved by the institutional review board at each site. Blood samples for analysis of serum biomarkers are obtained with consent from 30 age-appropriate individuals without neurological illnesses.

Eligibility criteria. Eligible subjects are 18 years of age and older with a diagnosis of Huntington's disease confirmed by genetic testing, a total functional capacity score of ≧5, and a caregiver to witness consent and monitor compliance. Exclusion criteria include previous creatine ascorbate exposure within 30 days of baseline; underlying hematological, hepatic, or renal disease; screening white blood cell count<3,800/mm³; creatinine>2.0 mg/dL or alanine aminotransferase greater than twice the upper normal limit; or unstable medical/psychiatric illness.

Study protocol. Subjects are screened within 25 days of randomization (baseline visit). Screening includes assessment of eligibility criteria, medical history, physical examination, Unified Huntington's Disease Rating Scale (UHDRS), EKG, DNA analysis, complete blood count, chemistry panel, and urinalysis. Subjects are on the study drug for 16 weeks, which will be followed by an 8-week washout. Study visits occur at baseline, weeks 8, 16, and 24 with telephone contacts at weeks 1, 10 and 20. Physical examinations are done at screening and week 16. UHDRS scores, vital signs, adverse event assessment, and safety laboratory tests are repeated at all visits. Blood is collected for serum creatine levels and serum 8-hydroxy-2′-deoxyguanosine (80H2′ dG), a measure of oxidative injury affecting DNA. The latter is analyzed in the samples from two sites. Magnetic resonance spectroscopy (MRS; STEAM; TR/TE=6000/20 milliseconds) of frontal cortex (voxel size=56 cc) and occipital cortex (voxel size=18 cc) is analyzed for this report. Subject compliance is assessed by wafer counts.

Statistical analysis. The primary outcome measure is tolerability. Subjects who do not complete week 16 or require more than one drug suspension or any suspension exceeding 7 days are considered treatment failures. In accordance with intent to treat, all randomized subjects are included in safety and tolerability analysis. Tolerability is assessed by comparing the proportion of treatment failures in creatine and placebo groups using χ² test with continuity correction. As the primary interest is in detecting intolerability in the active group, a one-sided test is used (significance=0.05). This was also applies to assess adverse events and laboratory abnormalities. The laboratory score changes from screening are analyzed by repeated-measures analysis of variance (ANOVA), and the differences between visits are analyzed by paired t test. Demographic and baseline variables are summarized for each group, and comparisons made using Fischer's exact test and continuous variables are compared using t test. Changes in UHDRS motor, cognitive, behavioral, and functional component subscores are measured at baseline and weeks 8, 16, and 24 and analyzed by mixed-model ANOVA.

### Example 2

### Randomized, Double-Blind, Futility Clinical Trial on Creatine Ascorbate and Minocycline in Early Parkinson's Disease (PD)

Methods

Study Design and Randomization. A single arm futility study is designed to assess the drugs creatine ascorbate and minocycline. Eligible subjects are randomly assigned in a 1:1:1 fashion to receive 1) 10 g/day of creatine ascorbate and placebo minocycline, 2) placebo creatine ascorbate and 200 mg/day of minocycline, or 3) placebo creatine ascorbate and placebo minocycline. The primary futility analysis is at 12 months of follow-up, but each subject is followed for 18 months for additional safety information. Subjects and investigators are kept blinded to treatment group.

Participants. Participants are men and women age 30 and over who had a diagnosis of PD but did not require medications for the management of their symptoms. Two of the three cardinal manifestations of PD (tremor, rigidity, and bradykinesia) are required; these findings are to be asymmetric. The diagnosis of PD must have been made within 5 years of randomization. Women of childbearing potential are required to use adequate birth control and have a negative pregnancy test at baseline. Subjects are excluded if they had any secondary causes of parkinsonism, such as drug induced parkinsonism or structural lesions; had atypical parkinsonian syndromes; gait freezing or impairment in postural reflexes; had prior stereotaxis surgery for PD; used creatine, minocycline, or any investigational agent within 90 days prior to randomization; had known hypersensitivity to creatine ascorbate or minocycline; used CoQ₁₀ in doses greater than 300 mg 90 days prior to randomization; or have any clinically significant medical condition that could interfere with the subject's ability to safely participate in the study or be followed.

Dosages. Creatine ascorbate is administered as 5 g sachets mixed with 8 ounces of liquid taken twice a day and minocycline is administered as 100 mg capsules taken twice a day. Both were taken with meals.

Outcome measures. The primary, prespecified outcome measure is the change in the total Unified PD Rating Scale (UPDRS) score from baseline to either the time at which there is sufficient disability to warrant symptomatic therapy for PD or 12 months, whichever came first. Disability is assessed by the sight investigator, based on impairment in ambulation, activities in daily living, and occupational status. The mean change in total UPDRS for each treatment group is compared to a prespecified futility threshold of a 30% reduction in the historically derived change in the total UPDRS, which is based on a placebo arm of a previous clinical trial. Tolerability is defined as the proportion of subjects taking study drug for the full 12 months. All severe adverse events (SAEs) are reviewed by the study medical monitor and an independent medical monitor. Both the site investigator and medical monitors assess the potential relationship between SAEs and study drug.

Study Procedures. At the screening visit, the purpose and potential risks are explained to potential subjects and each subject is given written consent. Subjects then have a baseline medical history, physical examination, and undergo the UPDRS. Blood is obtained for serum chemistry and complete blood count. Participants are reevaluated at 1, 3, 6, 9, and 12 months (±6 days) after the baseline visit using the battery of clinical scales and blood is drawn again at 6 and 12 months.

Sample size and statistical analysis. The sample size estimation is based on data from patients on placebo/tocopherol participating in the Deprenyl and Tocopherol Antioxidant Therapy of Parkinsonism trial (DATATOP), a large cohort of newly diagnosed patients with PD similar to the planned study population. The DATATOP study meets the Pocock criteria for the use of historical controls. As with most clinical studies, a certain degree of noncompliance (including subject withdrawl or lost-to-follow-up) is expected. Assuming the non-compliance rate to be minimal at 5%, the required sample size is increased to 65 per treatment arm to account for the noncompliance in the intent-to-treat analysis. For each study arm, the set of statistical hypotheses is as follows. H₀: Δᵢ≦7.46 vs Hₐ, Δᵢ>7.46 (=10.65), where Δᵢ is the mean change score (total UPDRS at 12 months or at the time of initiation of symptomatic therapy-total UPDRS at baseline) for the treatment arm i and 7.46 is the maximum mean increase (worsening) in the score between baseline and 12 months sufficient to warrant further evaluation of the drug in the Phase II trial. The hypothesis is tested with a one-sample t test at one-sided alpha level of 0.10. If the null hypothesis is rejected (p≦0.1) then the drug is considered futile for further testing in a Phase III trial.

A secondary analysis of the primary outcome is planned, if the mean change in the total UPDRS score observed in the calibration placebo group falls outside of the 95% CI of the historical control group mean change score of 10.65 (±1.02). The historical rate derived from DATATOP is updated by incorporating the information from the calibration placebo group using Bayesian methods to derive a posterior mean. The futility threshold is recomputed as 70% of this posterior mean and a one-sample t test is performed for each active treatment arm.

Analysis of the primary outcome is conducted under the intent-to-treat principle where all randomized subjects are included in the analyses. For the small proportion of subjects who are lost to followup, the UPDRS change scores are imputed using the worst change score observed within their respective treatment groups. Exploratory analyses include multiple imputation to account for missing values and a sensitivity analysis which used a 30% reduction from the observed calibration placebo value to recomputed the futility threshold value.

Subjects enrolled. Eligible subjects (200) are randomized to one of three treatment groups: Group 1 received creatine ascorbate; Group 2 received minocycline; Group 3 received placebo. The treatment groups were similar at baselines on demographic variables and total UPDRS and UPDRS subscores.

### Example 3

### High-Dose Creatine in Symptomatic Huntington's Disease

A two-phase open-label study is conducted to better determine an optimal dose of creatine ascorbate to symptomatic subjects with Huntington's disease. A dose-escalation study (10-40 grams per day) is conducted to determine the maximally tolerated dose (MTD) followed by a de-escalation phase to assess whether brain and serum levels of creatine might be maximal at doses lower than the MTD. Ten subjects are enrolled and followed prospectively for two weeks at each dose level increasing in 5-gram increments during dose escalation that lasts 13 weeks. Assessments at each visit include UHDRS, EKG, vital signs, clinical safety and research labs. MRI spectroscopy is conducted prior to baseline at peak done (40 grams) and one month after de-escalation to either 30 or 15 grams daily. To determine an optimal dose, pharmacokinetic as well as clinical data are considered. Once the maximal dose is reached, subjects are assigned one of two lower doses previously taken (15 grams a day (n=5) or 30 grams a day (n=5)). Serum creatine is assessed at baseline, at the end of each 2-week dose escalation step and at the end of the de-escalation phase to assess the correspondence between serum and brain levels of creatine at steady state.

Subjects at the end of this study are given the option to continue a long-term study to evaluate the long-term safety and tolerability of high dosage of creatine ascorbate. Subjects are followed for nine months on creatine ascorbate.

Morphometric neuroimaging is performed in all subjects. Longitudinal data from up to three years prior to initiating creatine is available on 6 or the 10 subjects. The rate of thinning of cortical regions in HD is modeled based on the longitudinal data and a change in rate is determined for each region of the group while on creatine ascorbate.

### EQUIVALENTS

Those skilled in the art will recognize, or be able to ascertain using no more than routine experimentation, many equivalents to the specific embodiments of the invention described herein. Such equivalents are intended to be encompassed by the following claims.

The entire contents of all references, patents, and patent applications cited herein are expressly incorporated by reference.

1. A composition comprising creatine bound to a ligand to form a creatine-ligand compound, wherein the creatine-ligand compound has a ratio of between about 1:1 creatine to ligand and about 10:1 creatine to ligand.

2-4. (canceled)

5. The composition of claim 1, wherein the ligand comprises an amino acid or a water-soluble vitamin.

6-8. (canceled)

9. The composition of claim 1, wherein the ligand is selected from the group consisting of cinnamate, lactate, glycolate, malate, mandelate, ascorbate, phytate, citrate, hydroxycitrate, aleurate, salicylate and hyaluronate.

10-20. (canceled)

21. A method of treating a creatine responsive state in a subject comprising administering to said subject a composition comprising an effective amount of a creatine-ligand compound such that the creatine responsive state in said subject is treated.

22. (canceled)

23. (canceled)

24. The composition of claim 21, wherein said creatine responsive state is a neurological disorder or a skin disorder.

25-51. (canceled)

52. The method of claim 21, further comprising administering to said subject an effective amount of an anti-inflammatory compound.

53. The method of claim 52, wherein said neurological disorder is Huntington's disease, Parkinson's disease, creatine transporter defect or amyotrophic lateral sclerosis.

54-60. (canceled)

61. The method of claim 52, wherein the anti-inflammatory compound is a member of the tetracycline family or a cyclooxygenase-2 (COX-2) selective inhibitor.

62-73. (canceled)

74. A pharmaceutical composition comprising an effective amount of a creatine-ligand compound and an acceptable carrier, wherein said effective amount is effective for the treatment of a creatine responsive state.

75. The pharmaceutical composition of claim 74, further comprising an effective amount an anti-inflammatory compound.

76-96. (canceled)

97. The method of claim 21, wherein said composition further comprises co-enzyme Q₁₀, ethyl-eicosapentanopic acid, a glutamate antagonist or phenylbutyrate.

98. (canceled)
